(19) United States
(12) Patent Application Publication
(10) Pub. No.: US 2025/0262249 A1
(43) Pub. Date: Aug. 21, 2025

(54) MULTI-PART PROCESSED HUMAN AMNIOTIC COMPOSITION AND METHODS OF MAKING AND USING THEREOF FOR TREATMENT OF PEYRONIE'S DISEASE

(71) Applicant: BioStem Technologies, Inc., Pompano Beach, FL (US)

(72) Inventors: Jason V. Matuszewki, Nashville, TN (US); Wendy W. Weston, Coral Springs, FL (US)

(21) Appl. No.: 18/858,536

(22) PCT Filed: Apr. 19, 2023
(86) PCT No.: PCT/US2023/019100

§ 371 Date: Oct. 21, 2024

Related U.S. Application Data
(60) Provisional application No. 63/332,749, filed on Apr. 20, 2022.

(51) Int. Cl. A61K 35/50

(52) U.S. Cl. CPC A61K 35/50

(57) ABSTRACT

A multi-part processed human amniotic composition configured to treat Peyronie's disease in a subject in need thereof by intracorporeal injection of the composition into the corpus cavernosum of the subject to reduce plaque size associated with Peyronie's disease. The multi-part processed human amniotic composition includes a micronized human amnion composition; and an aqueous human amniotic fluid filtrate configured to reconstitute and suspend the micronized human amnion composition therein. In certain aspects, the multi-part processed human amniotic compositions are not processed with exogenous enzymes during production thereof and do not include exogenous enzymes, such as collagenase, added thereto.

### TECHNICAL FIELD

The present invention relates generally to the field of amnion derived compositions, and more particularly, to non-immunogenic, multi-part processed human amniotic compositions for treatment of Peyronie's disease, which are directly produced from human amnion and amniotic fluid without the use of exogenous enzymes and/or do not include any added/exogenous enzymes such as collagenase.

### BACKGROUND

The human penis is an externally located component of the male reproductive system consisting of three chambers made up of sponge-like tissue surrounded by the tunica albuginea.

During sexual arousal, a physiological response occurs in which the penile tissue fills with blood perfused from elsewhere in the human body thereby increasing penis rigidity. Several penile disorders including erectile dysfunction (ED) and Peyronie's disease (PD) affect and/or hinder this physiological response. ED is generally defined as the inability to obtain and/or maintain an erection for sexual intercourse. It is a common condition, affecting about half of American men over 40. Causes of ED include vascular disorders, nerve disorders, psychological stresses, penile injury, chronic illness and unhealthy lifestyle habits.

In addition to the above mentioned causes of ED, ED sometimes results from Peyronie's disease. Peyronie's disease is the development of plaques (i.e., scar tissue) inside the tunica albuginea of the penis. When these plaques are high in number and/or cover a large surface area within the penis, curved, painful erections results, which often leads to ED making sexual intercourse very painful, difficult, and/or impossible.

Approximately 1 in 100 men in the United States over the age of 18 have been diagnosed with Peyronie's disease and the chance of developing Peyronie's disease increases with age. A significant number of men with PD develop erectile dysfunction. The exact cause of PD is unknown but is theorized to result from injury.

Scarring (plaques) may develop in the elastic layers that surround the erectile tissue reducing the elasticity of the penis in the area affected. Symptoms of PD include scar tissue that can be felt under the skin of the penis, a significant bend or curve of the penis, difficulty getting or keeping an erection, pain in the penis, and shortening of the penis. Surgical treatments include removing the plaque, shortening the tissue on the side opposite the plaque to even out the bend and penile implant. These procedures risk loss of erectile function or permanent shortening of the penis.

Non-surgical treatments utilize injections of various compositions to soften the plaques and correct the curvature. These compositions currently include XIAFLEX®, which is composed of collagenase derived from bacteria for the purpose of dissolving the plaque collagens. XIAFLEX® carries risk of corporal rupture, penile hematoma, ED and blood in the urine XIAFLEX®. Verapamil is a calcium channel inhibitor used to stop the progression of plaque formation. Side effects include dizziness, weakness, nausea and sweating. Verapamil also has a long list of drug interactions. Steroid injections have been used for many years. Many patients find the injections painful. The side effects of corticosteroids are reduction in immune system function, infection at the injection site, reduction in the size of penile tissue, thinning of the skin and complication of any future surgical procedures. Radiation therapy must be used in the early stages of PD to be effective and is used to stop the progression of PD. Studies for this approach lack solid controls and exposure of reproductive tissue to radiation can be a deterrent for many patients.

### SUMMARY

In view of the above problems existing with current Peyronie's disease treatments, a need exists for the treatment of penile defects (e.g., Peyronie's disease) that exhibit minimal side effects while achieving resolution of the penile defect. The compositions and methods disclosed herein achieve this objective by utilizing a multi-part processed human amniotic compositions disclosed herein as an intracorporeal injectable treatment for Peyronie's disease in subject's in need thereon, in which side effects and injection discomfort are minimized, while resolution of penile defects is maximized. Moreover, the disclosed multi-part processed human amniotic compositions are not processed with exogenous enzymes during production thereof and do not include added exogenous enzymes, such as collagenase, thereby preferably maintaining a similar endogenous profile observed in human amniotic fluid and human amnion tissue (e.g., in vivo), especially when compared with amnion isolates and/or other PD treatment formulations. By mimicking, including, and/or retaining a cellular and/or extracellular profile similar to the endogenous profile of a human amnion and human amniotic fluid (e.g., in vivo), the disclosed compositions advantageously minimize and/or completely eliminate immunogenic responses and/or other side effects observed in conventional Peyronie's disease treatments.

Disclosed herein are multi-part processed human amniotic compositions configured for treatment of Peyronie's disease by intracorporeal injection in a subject in need thereof with an effective amount of the multi-part process human amniotic composition. The multi-part processed human amniotic composition includes (a) a micronized human amnion composition; and (b) an aqueous human amniotic fluid filtrate configured to reconstitute and suspend the micronized human amnion composition therein, wherein the multi-part processed human amniotic composition is not processed with exogenous enzymes during production thereof and do not include exogenous enzymes (e.g., collagenase) added thereto.

In certain aspects, the micronized human amnion composition is a dried or milled micronized human amnion tissue.

In certain aspects, the micronized human amnion composition has a particle diameter size ranging from greater than 1 μm to less than 300 μm.

In certain aspects, the micronized human amnion composition has a particle diameter size ranging from greater than 1 μm to 50 μm, with the particles being polydisperse. In alternative aspects, the micronized human amnion composition has a particle diameter size ranging from greater than 1 μm to 50 μm, with the particles being substantially monodisperse.

In certain aspects, the micronized human amnion composition has a particle diameter size ranging from greater than 1 μm to 25 μm, with the particles being polydisperse. In alternative aspects, the micronized human amnion composition has a particle diameter size ranging from greater than 1 μm to 25 μm, with the particles being monodisperse.

In certain aspects, the micronized human amnion composition comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof.

In certain aspects, micronized human amnion composition is dried micronized human amnion tissue having a particle diameter size ranging from greater than 1 μm to less than 300 μm and comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof.

In certain aspects, the aqueous human amniotic fluid filtrate comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof.

In certain aspects, the aqueous human amniotic fluid filtrate further comprises an isotonic solution. In certain aspects, the isotonic solution is phosphate buffered saline.

In certain aspects, the aqueous human amniotic fluid filtrate comprises particles that are less than 70 μm in diameter therein.

In certain aspects, the multi-part processed human amniotic compositions includes HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, transthyretin ranging from 1.25×10⁴−2.25×10⁴ pg/mL, or any combination thereof when the micronized human amnion composition is reconstituted and/or suspended in the aqueous human amnion filtrate.

In certain aspects, the multi-part processed human amniotic compositions includes HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ pg/mL to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, and transthyretin ranging from 1.25×10⁴−2.25×10⁴ pg/mL when the micronized human amnion composition is reconstituted and/or suspended in the aqueous human amnion filtrate.

In certain aspects, the multi-part processed human amniotic compositions include the micronized human amnion composition and the aqueous human amniotic fluid filtrate configured for admixing at a ratio of 1 cm²: 1 ml to 2 cm²: 1 ml. In certain aspects, the multi-part processed human amniotic compositions include a cryopreservant therein and at a concentration of 1 to 10 wt % of the overall concentration of the multi-part processed human amniotic compositions. For example, in certain aspects, the cryopreservant may be dimethyl sulfoxide (DMSO) in the multi-part processed human amniotic composition at an overall concentration of 1 to 10 wt % of the of the multi-part processed human amniotic composition. Alternatively, the cryopreservant may be a DMSO-free cryopreservant (e.g., a polyampholyte, P24, etc.) at an overall concentration of 1 to 10 wt % of the multi-part processed human amniotic composition.

In certain aspects, both the micronized human amnion composition and the aqueous human amniotic fluid filtrate are sterile and/or aseptic.

In certain aspects, both the micronized human amnion composition and the aqueous human amniotic fluid filtrate are non-immunogenic.

In certain aspects, the multi-part processed human amniotic composition reduces size of Peyronie's disease plaques.

In certain aspects, transthyretin is present in the multi-part processed human amniotic composition at an effective amount to treat Peyronie's disease, reduce symptoms associated with Peyronie's disease, and/or an effective amount to reduces size of Peyronie's disease plaques.

In certain aspects, transthyretin is present in the multi-part processed human amniotic composition at a concentration ranging from 1.25×10⁴−2.25×10⁴ pg/mL.

Also disclosed herein are kits including the multi-part processed human amniotic composition configured for treatment of Peyronie's disease by intracorporeal injection in a subject in need thereof by administering an effective amount of the multi-part process human amniotic composition. The kit includes a sterile container having the multi-part processed human amniotic composition configured for treatment of Peyronie's disease by intracorporeal injection stored therein, wherein: the multi-part processed human amniotic composition is configured for treatment of Peyronie's disease by intracorporeal injection comprises micronized human amnion composition suspended in an aqueous human amniotic fluid filtrate, but does not include any exogenous enzymes added thereto.

In certain aspects, collagenase is not included in the multi-part processed human amniotic composition configured for treatment of Peyronie's disease by intracorporeal injection.

In certain aspects, the micronized human amnion composition is a dried or milled micronized human amnion tissue, preferably not subjected to exogenous enzymatic digestion.

In certain aspects, the micronized human amnion composition has a particle diameter size ranging from greater than 1 μm to less than 300 μm.

In certain aspects, the micronized human amnion composition has a particle diameter size ranging from greater than 1 μm to 50 μm, with the particles being polydisperse.

In certain aspects, the micronized human amnion composition has a particle diameter size ranging from greater than 1 μm to 25 μm, with the particles being polydisperse.

In certain aspects, the micronized human amnion composition comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof.

In certain aspects, the micronized human amnion composition comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), and transthyretin.

In certain aspects, the micronized amnion composition is dried micronized human amnion tissue having a particle diameter size ranging from greater than 1 μm to less than 300 μm and comprises hyaluronic acid (HA) and/or hyaluronan, fibronectin, insulin growth factor binding protein-1 (IGFBP-1), sulfated glycoaminoglycans (sGAGs), exosomes, interleukin-1 receptor antagonist (IL-1ra), hepatocyte growth factor (HGF), transthyretin, or any combination thereof.

In certain aspects, the aqueous human amniotic fluid filtrate comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof.

In certain aspects, the aqueous human amniotic fluid filtrate further comprises an isotonic solution.

In certain aspects, the isotonic solution is phosphate buffered saline.

In certain aspects, the aqueous human amniotic fluid filtrate comprises particles that are less than 100 μm in diameter therein.

In certain aspects, the micronized human amnion composition and the aqueous human amniotic fluid filtrate are admixed at a ratio of 1 cm²: 1 ml to 2 cm²: 1 ml In certain aspects, the multi-part processed human amniotic compositions include a cryopreservant therein and at a concentration of 1 to 10 wt % of the overall concentration of the multi-part processed human amniotic compositions. For example, in certain aspects, the cryopreservant may be dimethyl sulfoxide (DMSO) in the multi-part processed human amniotic composition at an overall concentration of 1 to 10 wt % of the of the multi-part processed human amniotic composition. Alternatively, the cryopreservant may be a DMSO-free cryopreservant (e.g., a polyampholyte, P24, etc.) at an overall concentration of 1 to 10 wt % of the multi-part processed human amniotic composition.

In certain aspects, the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ pg/mL to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, transthyretin ranging from 1.25×10⁴−2.25×10⁴ pg/mL, or any combination thereof.

In certain aspects, the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ pg/mL to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, and transthyretin ranging from 1.25×10⁴−2.25×10⁴ pg/mL.

In certain aspects, the multi-part processed human amniotic composition configured for treatment of Peyronie's disease is sterile and/or aseptic.

In certain aspects, the multi-part processed human amniotic composition configured for treatment of Peyronie's disease is non-immunogenic.

In certain aspects, the multi-part processed human amniotic composition configured for treatment of Peyronie's disease, to reduce symptoms associated with Peyronie's disease, and/or to reduce size of Peyronie's disease plaques.

In certain aspects, transthyretin is present in the multi-part processed human amniotic composition at an effective amount to treat Peyronie's disease, reduce symptoms associated with Peyronie's disease, and/or an effective amount to reduces size of Peyronie's disease plaques.

In certain aspects, transthyretin is present in the multi-part processed human amniotic composition at a concentration ranging from 1.25×10⁴−2.25×10⁴ pg/mL.

Also disclosed herein are methods of treating Peyronie's disease in a subject in need thereof by (a) providing the multi-part processed human amniotic composition configured for treatment of Peyronie's disease as disclosed herein; and (b) injecting the multi-part processed human amniotic composition configured for treatment of Peyronie's disease at an effective amount into a corpus cavernosum in the subject in need thereof thereby treating Peyronie's disease, reducing symptoms associated with Peyronie's disease, and/or reducing plaque size associated with Peyronie's disease in the subject in need thereof.

In certain aspects, the method includes administering/injecting into the subject the multi-part processed amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, transthyretin ranging from 1.25×10⁴−2.25×10⁴ pg/mL, or any combination thereof and an overall volume of 250 μL to 5 mL per administration/injection.

In certain aspects, the method includes administering/injecting into the subject the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, and transthyretin ranging from 1.25×10⁴−2.25×10⁴ pg/mL combination thereof and an overall volume of 250 μL to 5 mL per administration/injection.

Also disclosed herein are methods of making the multi-part processed human amniotic compositions configured for treatment of Peyronie's disease by intracorporeal injection in a subject in need thereof, the method including: (a) providing a fresh human amnion; (b) separating the human amnion from other human placental components; (c) rinsing the separated human amnion of step (b) with an alcohol; (d) washing the separated human amnion of step (c) with an isotonic solution; (e) drying separated human amnion of step (d) at ambient temperature for 2-6 hours in biosafety cabinet with circulating fan, or in a dehydrator for 15 minutes to 24 hours (at a temperature ranging 30° C. to 40° C.), thereby forming dried human amnion; (f) grinding the dried human amnion thereby forming a micronized human amnion composition having particle diameters ranging from 1 to 500 microns; (a′) providing fresh human amniotic fluid; (b′) filtering fresh human amniotic fluid through a 200 micron filter thereby forming a first human amniotic fluid filtrate; (c′) filtering the first human amniotic fluid filtrate through a 100 micron filter thereby forming a second human amniotic fluid filtrate; (d′) filtering the second human amniotic fluid filtrate through a 70 micron filter thereby forming a third human amniotic fluid filtrate (f′) optionally diluting the third human amniotic fluid filtrate in a predetermined amount of a balanced salt solution thereby forming a dilute third human amniotic filtrate; (i) mixing the micronized human amnion composition with either the third human amniotic fluid filtrate or the dilute third human amniotic filtrate thereby forming the multi-part processed human amniotic composition configured for treatment of Peyronie's disease by intracorporeal injection, wherein none of the steps include introduction of exogenous enzymes.

In certain aspects, the fresh human amnion is obtained from a subject and is subsequently subjected to steps (a)-(e) within 48 to 72 hours post-childbirth and/or caesarean section.

In certain aspects, the method further includes, between steps (a)-(c), removing any blood clots present within the human amnion.

In certain aspects, step (d) is repeated between one to five times by discarding the used isotonic solution, and providing new isotonic solution and again washing the human amnion with the new isotonic solution.

In certain aspects, the isotonic solution is phosphate buffered saline provided at a volume ranging from 300 mL to 1000 mL per washing step (d).

In certain aspects, a grinding tool configured to grind and/or mince the dried human amnion is used during step (f) and grinds the dried human amnion at a range of 40 to 200 revolutions per minute (RPM) until the dried human amnion has been ground thereby forming the micronized human amnion composition having particle diameters ranging from 1 to 500 microns.

In certain aspects, the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, transthyretin ranging from 1.25×10⁴−2.25×10⁴ pg/mL, or any combination thereof.

In certain aspects, the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration ranging from 1.0×10⁹ to 2.0×10⁹, IL-1ra ranging from 2×10⁴ to 1.5×10⁵ pg/mL, HGF ranging from 900 to 3000 pg/mL, and transthyretin ranging from 1.25×10⁴−2.25×10⁴ pg/mL.

In certain aspects, transthyretin is present in the multi-part processed human amniotic composition at an effective amount to treat Peyronie's disease, reduce symptoms associated with Peyronie's disease, and/or an effective amount to reduces size of Peyronie's disease plaques.

In certain aspects, transthyretin is present in the multi-part processed human amniotic composition at a concentration ranging from 1.25×10⁴−2.25×10⁴ pg/mL.

Additional features, aspects and advantages of the invention will be set forth in the detailed description, which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a schematic depiction of the steps included for making the micronized human amnion composition of the multi-part processed amniotic composition for the treatment of Peyronie's disease;

FIG. 2 is a schematic depiction of the steps included for making the aqueous human amniotic filtrate included in the multi-part processed human amniotic composition for the treatment of Peyronie's disease;

FIG. 3 is a schematic depiction of the steps included for mixing the micronized human amnion composition of FIG. 1 with the amniotic filtrate of FIG. 2 thereby forming the multi-part processed human amniotic composition having the desired concentration profile of HA, sGAGs, IGFBP-A, IL-1ra, HGF, and Transthyretin profile in a predetermined volume for intracorporeal administration and treatment of Peyronie's disease to a subject in need thereof;

FIG. 4 is a photograph of the multi-part processed human amniotic composition packaged in a sterile container according to Step (j) of FIG. 3 for the treatment of Peyronie's disease;

FIG. 5 is a graph depicting the concentration of HA in the multi-part processed human amniotic composition according to Step (j) of FIG. 3 for the treatment of Peyronie's disease;

FIG. 6 is a graph depicting the concentration of sGAGs in the multi-part processed human amniotic composition according to Step (j) of FIG. 3 for the treatment of Peyronie's disease;

FIG. 7 is a graph depicting the concentration of IL-1RA in the multi-part processed human amniotic composition according to Step (j) of FIG. 3 for the treatment of Peyronie's disease;

FIG. 8 is a graph depicting the concentration of HGF in the multi-part processed human amniotic composition according to Step (j) of FIG. 3 for the treatment of Peyronie's disease;

FIG. 9 is a graph depicting the concentration of IGF-BP1 in the multi-part processed human amniotic composition according to Step (j) of FIG. 3 for the treatment of Peyronie's disease;

FIG. 10 is a graph depicting the concentration of Transthyretin in the human amniotic filtrate (Step (e′) and/or or Step (f) of FIG. 2) before mixing with the micronized human amnion composition in Step (i) of FIG. 3 for the treatment of Peyronie's disease;

FIG. 11 is a graph depicting the overall concentration of Transthyretin in the multi-part processed human amniotic composition according to Step (j) of FIG. 3 for the treatment of Peyronie's disease;

FIG. 12 is a graph depicting the overall Exosome concentration in the multi-part processed human amniotic composition according to Step (j) of FIG. 3 for the treatment of Peyronie's disease;

FIGS. 13A and 13B depict microscopic images with DAPI staining of the multi-part processed human amniotic composition using a 50 micron scale; FIGS. 13C and 13D depict microscopic images with DAPI staining of the multi-part processed human amniotic composition using a 20 micron scale; FIGS. 13E and 13F depict microscopic images with DAPI staining of the multi-part processed human amniotic composition using a 10 micron scale; and

FIG. 14 depicts exemplary endogenous cellular pathways activated by the multi-part processed human amniotic composition for the treatment of Peyronie's disease.

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein.

The exemplary embodiments are provided so that this disclosure will be both thorough and complete, and will fully convey the scope of the invention and enable one of ordinary skill in the art to make, use and practice the invention. Like reference numbers refer to like elements throughout the various drawings. Moreover, in this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

The compositions and methods described herein can comprise, consist of, or consist essentially of the essential elements and limitations described herein, as well as any additional or optional ingredients, components, or limitations described herein.

The medical benefits of human placental membranes have become increasingly apparent over the past several years, and as a result a variety of membrane combinations have been developed for applications ranging from cosmetic to invasive surgery.

With the above general advantageous medical benefits in mind of human placental membranes and in view of the endogenous components included in human amnion and human amniotic fluid, it was envisioned that a multi-part processed human amniotic composition for treatment would be useful for the treatment of Peyronie's disease, to reduce symptoms associated with Peyronie's disease, reduce plaque size associated with Peyronie's disease, improve penile function in subjects having Peyronie's disease, and restore penile function in subjects having Peyronie's disease. Generally speaking, human amniotic fluid (also referred to herein as “amniotic fluid” or “AF”) is a clear, slightly yellowish liquid that surrounds the fetus during pregnancy. AF contains nutrients and growth factors that facilitate fetal growth, provides mechanical cushioning and antimicrobial factors that protect the fetus. In addition, AF further includes proteins, electrolytes, immunoglobulins, and vitamins therein. Furthermore, AF contains high levels of sulfated glycosaminoglycans, IGFBP-1, and hepatocyte growth factor (HGF). Moreover, the amniotic fluid, as well as the amniotic membrane, includes transthyretin.

The human amnion (also referred to herein as “amnion”) is between 0.02 and 0.05 mm thick. The human amnion is avascular with no nerve or lymph, and in utero, receives nutrients through diffusion. The amnion is in direct contact with the amniotic fluid. The human amnion has three layers: the epithelial layer, the thick basement membrane, and the avascular mesenchymal tissue. These contain type I and type III-VII collagen and high concentrations of proteoglycans and glycoproteins along with fibronectin and laminin. The functions of amniotic membrane include physical protection of the fetus, protection from bacterial infection, regulation of pH, and secreting growth factors and other molecules. These serve antimicrobial and anti-inflammatory functions. Furthermore, the instant Applicants have observed that the amnion includes high levels of hyaluronic acid (HA) and IL-1ra.

In the context of penile defects (e.g., Peyronie's disease) and penile defect resolution, the amniotic membrane provides stromal support, ECM repair and anti-inflammatory factors. In the context of penile defects, AF provides hydration, stromal support and matrix remodeling factors. Moreover, the amniotic fluid includes transthyretin.

Although human amnion and human amniotic fluid have little medical and/or therapeutic use in its natural state, the above mentioned endogenous components included in human amnion and human amniotic fluid when further processed into a medicament having the proper concentrations of the above discussed components and volumes can be used to treat Peyronie's disease (via intracorporeal injection/administration) by reducing plaque size, promoting endogenous remodeling of the corpus cavernosum, improving ECM characteristics of the corpus cavernosum, reducing inflammation within the corpus cavernosum and other penile tissues, and improving/restoring functions other penile tissues to restore normal sexual function.

### Multi-Part Processed Amniotic Composition

With the above in mind, disclosed herein are multi-part processed amniotic compositions derived from human amnion and amniotic fluid, and when each component is mixed with one another, the resulting composition mimics, includes, and/or retains a cellular and/or extracellular profile similar to the endogenous profile of a human amnion and/or amniotic fluid, for example, in vivo, especially when compared with isolates thereof. Moreover and to further maximize the retention of the cellular and/or extracellular profile similar to the endogenous profile of a human amnion and/or amniotic fluid, for example, in vivo, these compositions are not processed with exogenous enzymes during production. Furthermore, these compositions do not include any added enzymes, such as collagenase, in the final end product (i.e., multi-part processed amniotic compositions) thereby advantageously minimizing and/or eliminating immunogenic responses and/or other side effects observed in conventional Peyronie's disease treatments.

In particular, the disclosed compositions are prepared with fresh human amnion and amniotic fluid (harvested and processed within 0 to 72 hours post extraction from the human subject) and are preferably maintained at from 4° C. to 8° C. during harvesting and processing while making the multi-part processed amniotic compositions to prevent and/or reduce any endogenous enzymatic degradative processes. Likewise, the disclosed multi-part processed amniotic compositions are subsequently stored at temperatures ranging from 4° C. to 8° C. to maintain preferred shelf life and to minimize degradation of these composition while stored.

Unlike compositions in the prior art, the disclosed compositions are advantageously not subjected to biochemical and/or enzymatic digestion, which results in the compositions including and/or retaining a significant portion of the cellular and/or extracellular profile (when compared to the endogenous profile of a human umbilical cord in vivo). Moreover because of the ease and convenience of preparing (sterile and/or aseptic preparation) these compositions (e.g., point of use preparation and use within a urologist's office or other medical facility) and because of the non-immunogenic characteristics of these compositions, these compositions may be used (e.g., as an injectable composition) within humans to advantageously treat Peyronie's disease and/or reduce plaque size in those afflicted with Peyronie's disease.

The multi-part processed human amniotic compositions configured for treatment of Peyronie's disease by intracorporeal injection in a subject in need thereof with an effective amount of the multi-part process human amniotic composition. The multi-part processed human amniotic composition particularly includes (a) a micronized human amnion composition; and (b) an aqueous human amniotic fluid filtrate configured to reconstitute and suspend the micronized human amnion composition therein, wherein the multi-part processed human amniotic composition are not processed with exogenous enzymes during production thereof and do not include exogenous enzymes added thereto. The exclusion of exogenous enzymes during production of these compositions avoids undesired enzymatic degradation/digestion of the micronized human amnion compositions and the aqueous human amniotic fluid filtrate within the multi-part processed human amniotic compositions and further ensures that these compositions have an improved endogenous cellular and extracellular profile (similar to human amnion and amniotic fluid in vivo) especially when compared to conventional compositions utilizing amnion and amniotic fluid and/or cell isolates derived therefrom.

In certain aspects, the micronized human amnion composition is a dried and/or milled (e.g., cryomilled) human amnion that was not subjected to exogenous enzymatic digestion during the preparation thereof, which further preserves and/or reduces degradation of endogenous hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof. For the reasons discussed further below, these components advantageously play a role in reducing plaque size associated with Peyronie's disease and/or further promoting endogenous remodeling of the corpus cavernosum and other penile tissues to restore normal sexual function.

The micronized human amnion composition of the multi-part process human amniotic composition are either polydisperse or monodisperse and have a particle diameter size ranging from greater than 1 μm to less than 500 μm, from greater than 1 μm to less than 300 μm, from greater than 1 μm to 100 μm, greater than 1 μm to 50 μm, or greater than 1 μm to 25 μm so that adequate mixing, wetting, reconstitution, dispersion, and/or suspension of the micronized human amnion composition occurs when mixed with the aqueous human amniotic fluid filtrate. It is further envisioned that any endpoint falling within the above-disclosed ranges may serve as endpoints for additional ranges. It is preferred that the micronized human amnion compositions have particle diameters falling within the above mentioned ranges so that homogeneous mixing, dispersion, wetting, and suspension is observed in the multi-part processed human amniotic compositions (when the micronized human amnion compositions are mixed with the aqueous human amniotic fluid filtrate forming the multi-part processed human amniotic compositions) thereby leading to increased efficacy of treatment when administered to the subject in need thereof because hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), and transthyretin within the multi-part processed human amniotic compositions have greater bioavailability while concurrently minimizing side-effects and/or discomfort associated with injectable compositions.

In certain aspects and when micronized human amnion composition particle diameters exceed 500 μm, poor mixing, poor wetting, non-homogeneous dispersion, and/or poor suspension is observed, which ultimately leads to either an inoperable and/or suboptimal compositions having poor bioavailability of the hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), and transthyretin in the micronized human amnion compositions (having diameters exceeding 500 μm) leading to decreased therapeutic effects for treatment of Peyronie's disease and/or have increased side-effects and/or discomfort during administration.

As alluded to above, the multi-part processed human amniotic compositions further include aqueous human amniotic fluid filtrate that is configured to reconstitute and/or suspend the micronized human micronized amnion composition. The aqueous human amniotic fluid filtrate is preferably prepared from the same human donor as the amnion (and as the micronized human amnion composition (disclosed above)) via one or more separation steps (e.g., filtration steps discussed further below). In certain aspects, the aqueous human amniotic fluid filtrate comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof at concentrations sufficient to treat Peyronie's disease and/or reduce Peyronie's disease plaque size when included in the multi-part processed human amniotic composition. In certain alternative aspects, the aqueous human amniotic fluid filtrate may be further diluted before being included in the multi-part processed human amniotic compositions to obtain hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof at concentrations sufficient to treat Peyronie's disease and/or reduce Peyronie's disease plaque size when included in the multi-part processed human amniotic composition. When diluted, the aqueous human amniotic fluid filtrate is diluted in an isotonic solution such as phosphate buffered saline (or one of lactated ringers (NaCL 6 g/L, Sodium Lactate 3.1 g/L, KCl 0.3 g/L, and CaCl 0.2 g/L at pH 6.5), isotonic saline (0.9 wt % NaCl), plasmalyte® (NaCl 5.26 g/L, KCI 0.37 g/L, Magnesium Chloride hexahydrate 0.30 g/L, Sodium Acetate trihydrate 3.68 g/L, Sodium Gluconate 5.02 g/L at pH 7.4)).

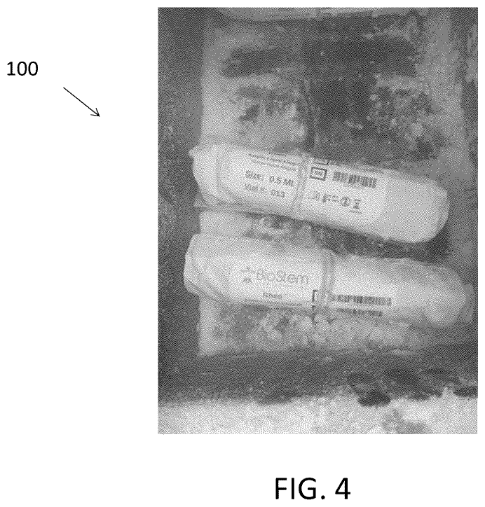

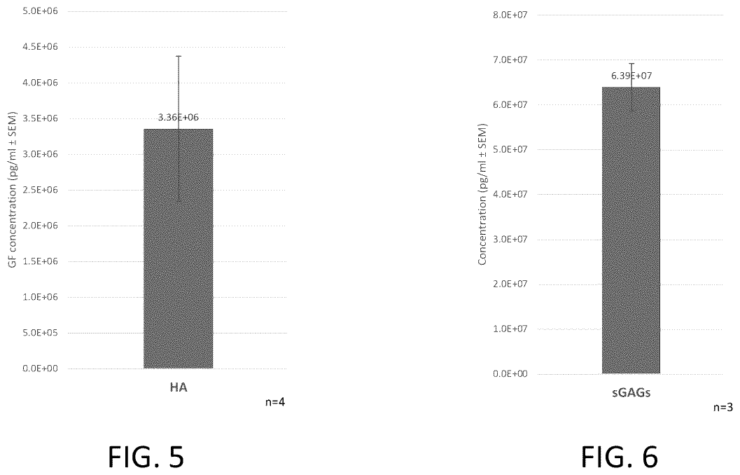

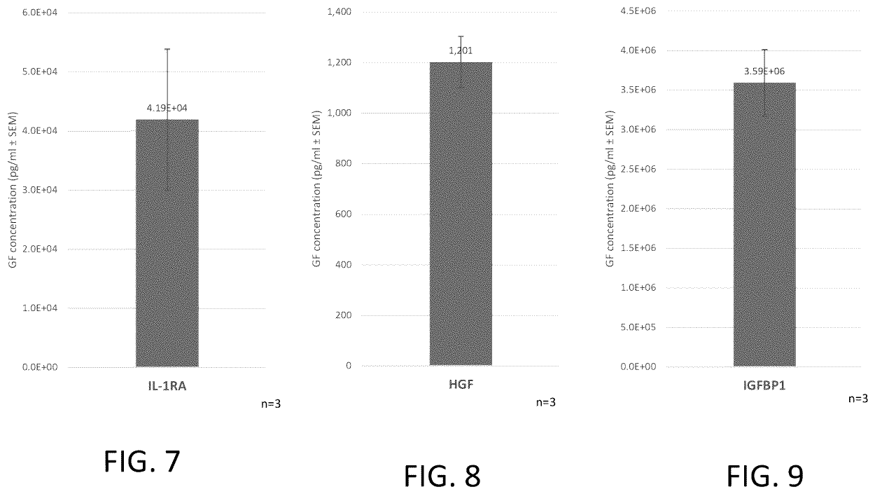

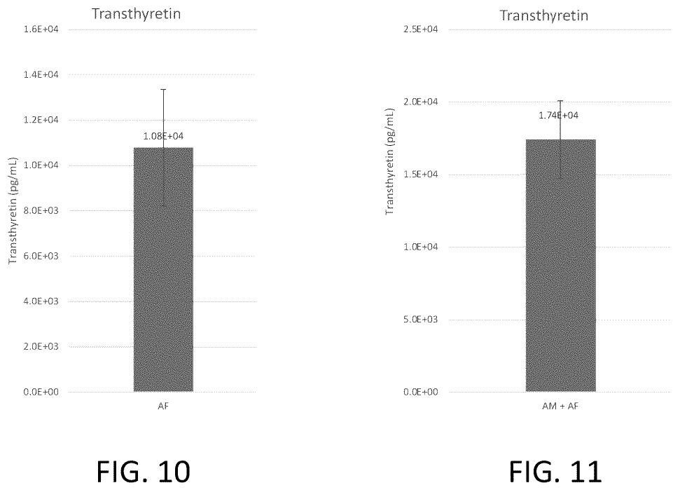

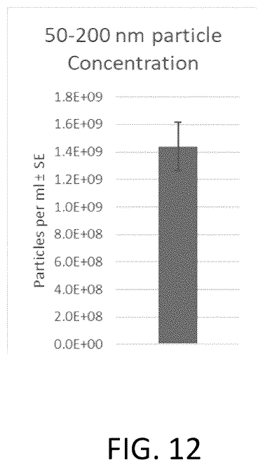

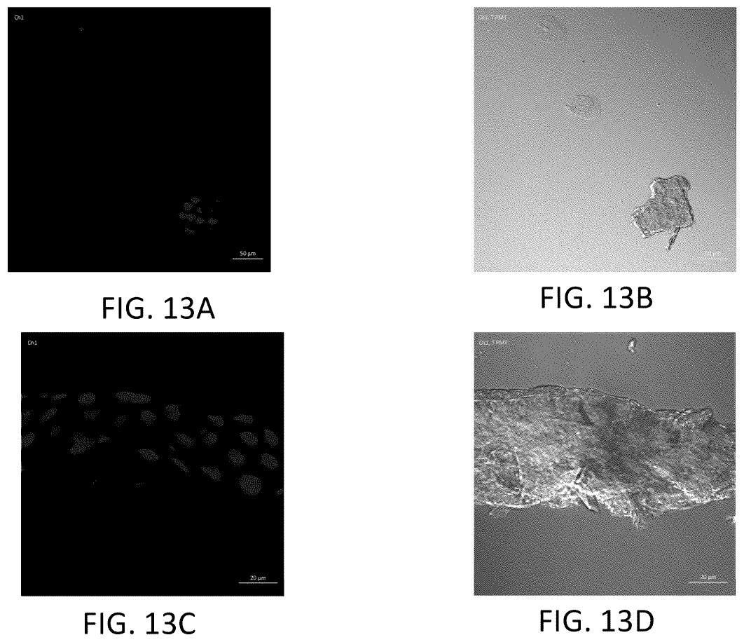

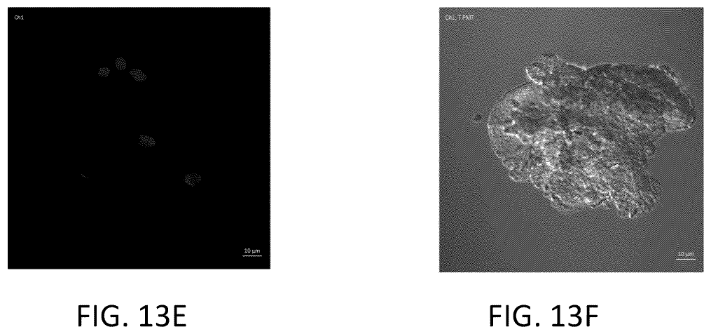

FIG. 4 is a photograph of the multi-part processed human amniotic composition packaged in an exemplary sterile container (100), stored at between −20° C. to −80° C. and ready for subsequent use. Other sterile containers may include ampules, pre-loaded syringes, and/or vials also stored at between −20° C. to −80° C. and ready for subsequent use. FIGS. 13A and 13B depict microscopic images with DAPI staining of the multi-part processed human amniotic composition using a 50 micron scale; FIGS. 13C and 13D depict microscopic images with DAPI staining of the multi-part processed human amniotic composition using a 20 micron scale; and FIGS. 13E and 13F depict microscopic images with DAPI staining of the multi-part processed human amniotic composition using a 10 micron scale. Moreover, FIGS. 5-9, 11, and 12 show the general concentration profiles for HA, SGAGs, IL-1RA, HGF, IGF-BP1, Transthyretin, and exosomes included in the multi-part processed human amniotic compositions (i.e., compositions having the micronized human amnion composition and the aqueous human amniotic fluid filtrate) configured for treatment of Peyronie's disease by intracorporeal injection in a subject in need thereof. While, in contrast to FIG. 11, FIG. 10 depicts transthyretin in only the aqueous human amniotic filtrate (i.e., excluding the micronized human amnion composition). In certain aspects and as shown in FIGS. 5-9, 11 and 12, the multi-part processed human amniotic compositions includes HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, transthyretin ranging from 1.25×10⁴−2.25×10⁴ pg/mL and/or ranging from 1.3×10^{4 ×to} 2.15×10⁴ pg/mL, or any combination thereof when the micronized human amnion composition is reconstituted and suspended in the aqueous human amnion filtrate. While in certain aspects, the multi-part processed human amniotic compositions includes HA at a concentration ranging from HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, and transthyretin ranging from 1.25×10⁴ to 2.25×10⁴ pg/mL and/or ranging from 1.3×10⁴ to 2.15×10⁴ pg/mL when the micronized human amnion composition is reconstituted and suspended in the aqueous human amnion filtrate. In certain aspects, both the micronized human amnion composition and the aqueous human amniotic fluid filtrate are sterile and/or aseptic, and the multi-part processed human amniotic compositions is sterile and/or aseptic as well. In certain aspects, both the micronized human amnion composition and the aqueous human amniotic fluid filtrate are non-immunogenic, and the multi-part processed human amniotic compositions is non-immunogenic as well. In certain aspects, treatment (i.e., intracorporeal injection/administration to a subject in need thereof) of the multi-part processed human amniotic composition treats Peyronie's diseases, reduces size of Peyronie's disease plaques, reduce symptoms associated with Peyronie's disease, improves penile function especially aiding in achieving a non-painful erection, and/or restores penile function such as achieving a non-painful erection.

In view of the above and without wishing to be bound by theory, transthyretin is the primary active ingredient/therapeutic agent for reducing plaque size associated with Peyronie's disease, treating Peyronie's disease, reducing symptoms of Peyronie's disease, and/or restoring and/or improving penile function in those suffering from Peyronie's disease. Transthyretin is present in the multi-part processed human amniotic composition at an effective amount to treat Peyronie's disease, reduce symptoms associated with Peyronie's disease, and/or an effective amount to reduce size of Peyronie's disease plaques, and in certain aspects, transthyretin is present in the multi-part processed human amniotic composition at a concentration ranging from 10 to 50 pg/mL. Although the mechanism of action has not been elucidated, transthyretin was previously shown to bind to beta-amyloid proteins (associated with early stages of Alzheimer's) thereby preventing beta-amyloid's natural tendency to accumulate into plaques. It is thought that the mode of action for reducing plaque size associated with Peyronie's disease (as well as treating Peyronie's disease, reducing symptoms of Peyronie's disease, and/or restoring and/or improving penile function in those suffering from Peyronie's disease) may be similar.

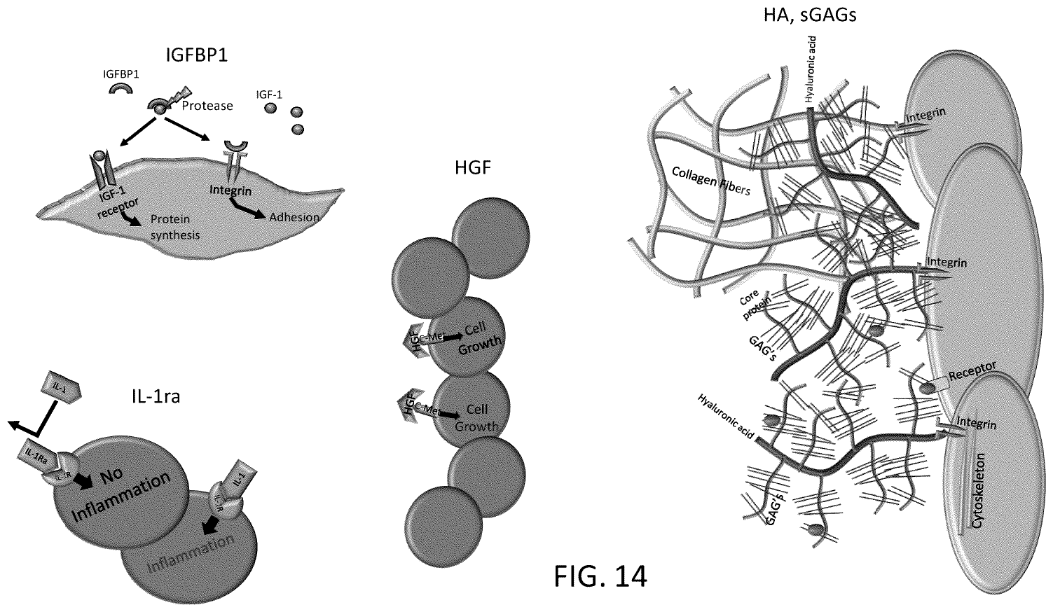

In addition to the inclusion transthyretin in the compositions as discussed above, the presence and concentrations disclosed above of HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, and transthyretin ranging from 1.25×10⁴ to 2.25×10⁴ pg/mL and/or ranging from 1.3×10⁴ to 2.15×10⁴ pg/mL in the multi-part processed human amniotic compositions play further roles in penile tissue remodeling and restorative function, which aids in treating and improving penile function in those suffering from Peyronie's disease. In particular, FIG. 14 schematically depicts the endogenous modes of action for IL-1RA, IGF-BP1, HGF, HA, and sGAGs. Hyaluronic acid (HA) is a chief component of the extracellular matrix [Toole 2001]. HA is a non-sulfated glycosaminoglycan, which, among other things, provides a backbone for sulfated glycosaminoglycans. HA also binds integrins resulting in stabilized ECM and absorbs water which is another chief component of the ECM. The high molecular mass of HA results in unique biophysical properties, such as, high viscoelasticity and high colloid osmotic pressure [Laurent 1995] [Cowman and Matsuoka 2005]. Additionally, HA leads to extracellular matrix stabilization, water maintenance and regulation of protein distribution. Sulfated glycosaminoglycans (sGAGs) interact with other ECM components to organize and form a hydrophilic matrix suitable for remodeling [Couchman 2012] [Prydz 2000]. sGAGs also bind to the HA backbone and carry factors to the tissues. IGFBP-1 is the predominant IGF-binding protein in amniotic fluid, fetal and maternal circulation [Hills 1996] [Hills 1996]. IGFBP1 binds IGF's to prolong their half-life and alters their interaction with cell surface receptors. IL-1ra binds non-productively to the IL-1 (proinflammatory cytokine) receptor, preventing IL-1 (inflammatory molecule) from sending a signal. This results in modulation of a variety of IL-1 related immune and inflammatory responses. HGF is cellular growth, motility and morphogenic factor secreted by mesenchymal cells. It has been shown to have a major role in embryonic organ development, specifically in myogenesis, in adult organ regeneration, and in wound healing. HGF has been associated with the enhanced and scarless wound healing capabilities of fibroblast cells isolated from the oral mucosa tissue [Dally 2017] and HGF, in the disclosed concentrations herein may play a role in reducing Peyronie's plaque size (in addition to the therapeutic effects and plaque reducing function of transthyretin).

### Method of Making Multi-Part Processed Amniotic Composition

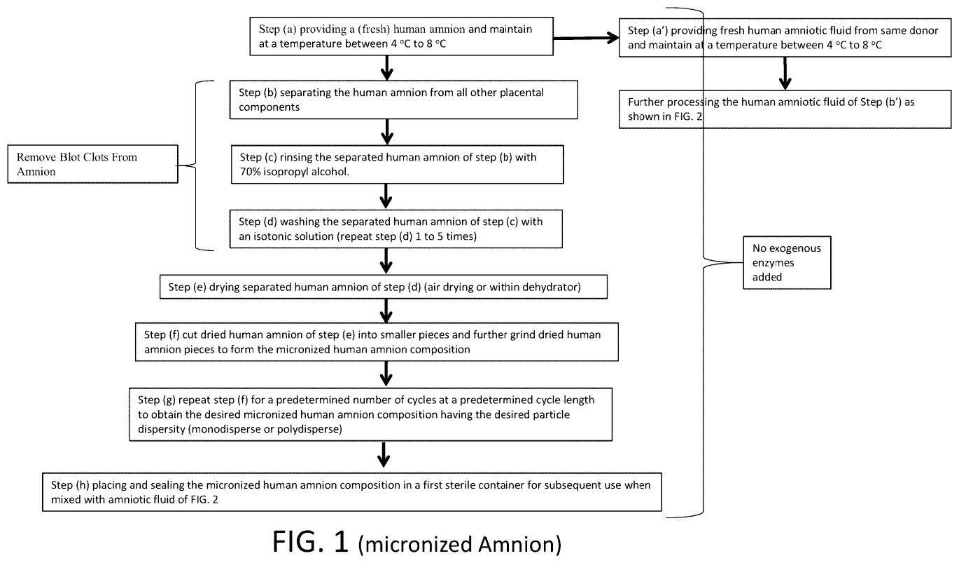

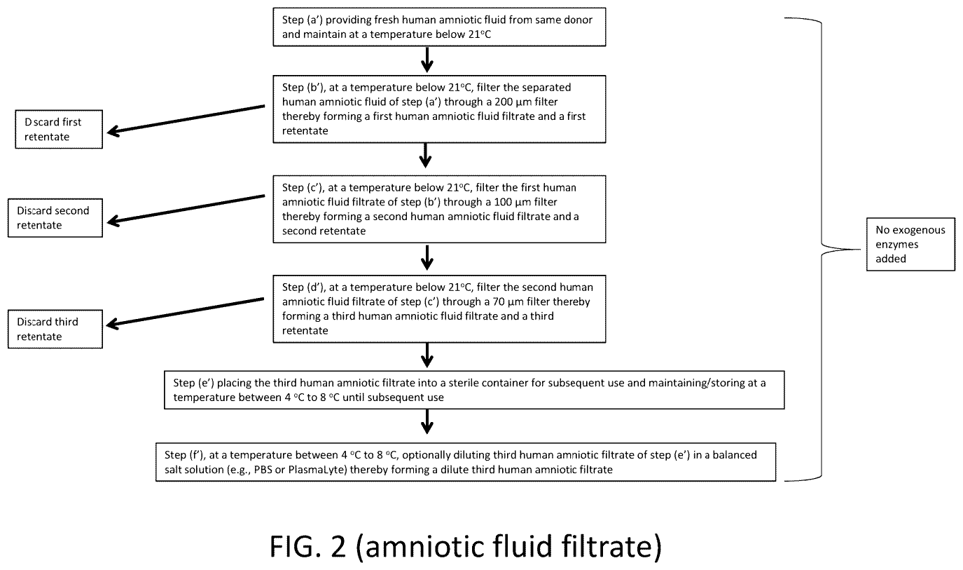

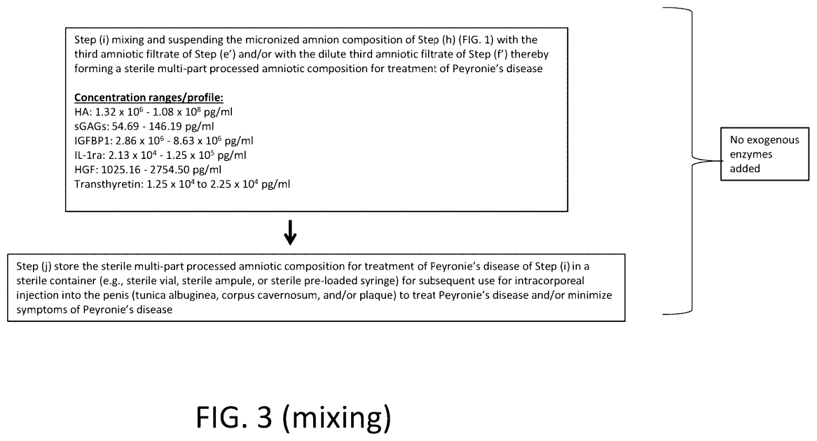

FIGS. 1-3 provide schematic depictions of the steps included for respectively making the micronized human amnion composition, the aqueous human amniotic filtrate, and the multi-part processed human amniotic compositions by mixing and/or suspending the micronized human amnion composition in the aqueous human amniotic filtrate. More particularly, FIG. 1 is a schematic depiction of the steps included for making the micronized human amnion composition of the multi-part processed amniotic composition for the treatment of Peyronie's disease. FIG. 2 is a schematic depiction of the steps included for making the human amniotic filtrate included in the multi-part processed human amniotic composition for the treatment of Peyronie's disease, and FIG. 3 is a schematic depiction of the steps included for mixing the micronized human amnion composition of FIG. 1 with the amniotic filtrate of FIG. 2 thereby forming the multi-part processed human amniotic composition having the desired concentration profile of HA, sGAGs, IGFBP-A, IL-1ra, HGF, and Transthyretin profile in a predetermined volume for intracorporeal administration and treatment of Peyronie's disease to a subject in need thereof.

With specific reference to FIG. 1, disclosed therein are the steps for making/preparing the micronized human amnion composition having the desired particle size/diameter. However, before step (a), the human amnion (as well as the human amniotic fluid) is screened for communicable diseases to ensure that the amnion tissue is healthy/disease free and to further minimize risk during preparation and subsequent end use of the multi-part processed human amniotic composition for the treatment of Peyronie's disease. After confirming that the human amnion tissue (as well as the human amniotic fluid) is healthy/disease free, the human amnion is maintained at temperature ranging from 4° C. to 8° C. before the processing of the amnion as shown in steps (a)-(g) in FIG. 1. As shown in FIG. 1, step (a) includes providing fresh human amnion and step (a′) includes providing fresh human amniotic fluid (preferably from the same donor) within 24 to 96 hours post-extraction from a human subject, more preferably from 24 to 72 hours post-extraction from a human subject to ensure freshness of the human amnion (i.e., tissue and cells comprising the tissue) as well as the human amniotic fluid to minimize degradation associated (enzymatic degradation) resulting from necroptosis and/or apoptosis.

During step (b), the human amnion is separated from other human placental components thereby forming the separated human amnion, and the separated human amnion of step (b) may be re-sized as desired during this step before proceed to step (c). For example, in certain aspects, it may be preferred to re-size the separated human amnion into 15 to 80 gram portions and more preferably 30 to 60 gram portions for ease of handling and subsequent processing in later steps.

After step (b), the separated human amnion is rinsed/with an alcohol solution during step (c) to rinse away an extraneous non-amnion human placental components and/or any other potential contaminants. In certain aspects, the alcohol solution preferably is isopropyl alcohol at a concentration of 70% to 100%. In preferred embodiments the alcohol concentration ranges from 70% to 75%, and in most preferred embodiments, the alcohol is 70% isopropyl alcohol. Isopropyl alcohol is preferred over other commercially available lab and/or pharmaceutical grade alcohols, such as ethanol, because isopropyl alcohol advantageously disinfects and cleans the human amnion without damaging (e.g., unduly dehydrating, initiating apoptotic processes, and/or necrotic processes) the amniotic tissue.

After step (c), the rinsed human amnion of step (c) is then washed in an isotonic solution during step (d). In particular, step (d) includes placing one or more human amnion portions (15 to 80 gram) into a container having a predetermined volume (e.g., 300 mL to 1000 mL, preferably 500 mL) of isotonic solution in which the isotonic solution is preferably phosphate buffered saline (PBS) (i.e., 1× PBS) (or alternatively one of lactated ringers (NaCL 6 g/L, Sodium Lactate 3.1 g/L, KCl 0.3 g/L, and CaCl 0.2 g/L at pH 6.5), isotonic saline (0.9 wt % NaCl), plasmalyte® (NaCl 5.26 g/L, KCI 0.37 g/L, Magnesium Chloride hexahydrate 0.30 g/L, Sodium Acetate trihydrate 3.68 g/L, Sodium Gluconate 5.02 g/L at pH 7.4)). Placing the container onto a stir plate and placing a stir bar within the container (containing the PBS and human amnion portions) therein and stirring (medium to high speed) the human amnion portions within the isotonic solution for 5 to 15 minutes to wash the human amnion portions. In certain aspects, washing step (d) is repeated one to five times by decanting the “used” isotonic solution and pouring new isotonic solution into the container at a predetermined volume (e.g., 300 mL to 1000 mL, preferably 500 mL) to again wash the one or more human amnion portions.

As further shown in FIG. 1, either before step (b), during step (b), before step (c), during step (d), before step (d), or during step (d) one should further determine whether any blood clots and/or blood pool(s)/pooling are present in the human amnion and/or amnion portions, and if so, removing these blood clots via suction or other mechanical removal means (e.g., scalpel and forceps) to further ensure that the presence of any immunogenic components (e.g., hemoglobin and/or heme associated components from the human amnion donor) are minimized in the end resulting micronized human amnion composition as well as the end resulting multi-part processed human amniotic composition. During these washing steps, it is imperative to maintain an aseptic and/or sterile work environment to prevent and/or reduce introduction of any contaminants while making micronized human amnion composition as well as the end resulting multi-part processed human amniotic composition.

After step (d), the washed human amnion from step (d) is then subjected to drying during step (e) thereby forming dried human amnion. In particular, during step (e), the amnion is laid flat and set at ambient temperature for 2-6 hours in the biosafety cabinet with circulating fan, or in a dehydrator for 15 minutes to 24 hours (at a temperature ranging 30° C. to 40° C.).

After step (e), the dried human amnion may be further re-sized into smaller portions and/or subjected to a grinding process. In particular, during step (f), the dried human amnion is subjected to a milling and/or cryomilling process configured to yield particles (polydisperse or monodisperse particles) having sizes ranging from greater than 1 μm to 500 μm, greater than 1 μm to 400 μm, more preferably greater than 1 μm to 300 μm, greater than 1 μm to 200 μm, greater than to than 1 μm to 100 μm, greater than to than 1 μm to 50 μm, greater than to than 1 μm to 25 μm greater than 50 μm to 400 μm, than 50 μm to 300 μm, than 50 μm to 200 μm wherein any endpoint falling within these ranges may serve as endpoints for additional ranges. In certain aspects, step (f) is a cryomilling process (as described, for example, US 20160287749, US 20170203004, and US Pat. No. 10,105,398, which are each incorporated by reference in their entirety herein) in which the dried human amnion of step (e) is placed into a liquid nitrogen cooled cryomill chamber and subjected to grinding therein thereby forming the micronized human amnion composition having the above mentioned particle diameter(s). In certain aspects, each grinding and/or milling (cryomilling) cycle is for a duration of 1 to 4 minutes, of 1 to 3 minutes, of 1.5 to 2.5 minutes. As further shown in FIG. 1, the method for producing the micronized human amnion composition may further include step (g), which is repeating the grinding and/or milling process of step (f) for an additional number of predetermined cycles (e.g., 1 additional cycle, 2 additional cycles, 3 additional cycles, 4 additional cycles, 5 additional cycles, 6 additional cycles, etc.) in which each additional cycle has a predetermined time duration (e.g., 1 to 4 minutes, of 1 to 3 minutes, of 1.5 to 2.5 minutes). These additional cycles result in a smaller and more homogeneous particle diameter of the micronized human amnion composition. As alluded to above, the micronized human amnion composition(s) of step(s) (f) and/or (g) include hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof at effective amounts/concentrations when mixed and/or re-suspended in the aqueous human amnion filtrate thereby forming the multi-part processed amniotic composition to treat Peyronie's disease, reduce symptoms associated with Peyronie's disease, reduce plaque size associated with Peyronie's disease in the subject in need thereof, and/or improve and/or restore penile function.

Each of steps (a)-(g) are preferably conducted in a sterile and/or aseptic environment and result in sterile micronized human amnion compositions, which is also non-immunogenic. Each of the steps (a)-(h) are conducted below 21° C. to prevent and/or reduce any endogenous enzymatic degradative processes during production of the micronized human amnion compositions and to maintain the desired hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), and/or transthyretin profiles therein.

After Step (f) or (g), the micronized human amnion composition may be optionally placed and sealed in a first sterile container (step (h)) for subsequent use/mixing and/or suspension in the aqueous human amniotic filtrate. Alternatively, step (h) may be omitted and the micronized human amnion composition may be immediately mixed and/or suspended in the aqueous human amniotic filtrate as shown in step (i) in FIG. 3.

FIG. 2 is a schematic depiction of the steps included for making the human amniotic filtrate included in the multi-part processed human amniotic composition for the treatment of Peyronie's disease. The steps shown in FIG. 2 may occur before, during, or after steps (a)-(h) of FIG. 1. As shown in FIG. 2, during step (a′) fresh human amniotic fluid is provided from a healthy/disease free donor within 0 hours to 72 hours of extraction/removal from the donor and maintained at temperature ranging from 4° C. to 8° C.

Next, during step (b′), the fresh human amniotic fluid is filtered through a 200 micron filter thereby forming a first human amniotic fluid retentate (first retentate) and a first human amniotic fluid filtrate. The first retentate is subsequently discarded. Next, during step (c′) the first human amniotic fluid filtrate is filtered through a 100 micron filter thereby forming a second human amniotic fluid retentate (second retentate) and a second human amniotic filtrate. The second retentate is subsequently discarded. Next, during step (d′) the second human amniotic filtrate is filtered through a 70 micron filter thereby forming a third human amniotic fluid retentate (third retentate) and a third human amniotic fluid filtrate, the third human amniotic filtrate is acellular, only includes any particulates therein that are less than 70 microns, and is sterile. The third human amniotic fluid filtrate includes hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof therein.

Next, the third human amniotic fluid filtrate is optionally subjected to step (f) in which the third human amniotic fluid filtrate is diluted in a predetermined amount of a balanced salt solution/isotonic solution (e.g., phosphate buffered saline (PBS) (i.e., 1× PBS) (or alternatively one of lactated ringers (NaCL 6 g/L, Sodium Lactate 3.1 g/L, KCl 0.3 g/L, and CaCl 0.2 g/L at pH 6.5), isotonic saline (0.9 wt % NaCl), plasmalyte® (NaCl 5.26 g/L, KCI 0.37 g/L, Magnesium Chloride hexahydrate 0.30 g/L, Sodium Acetate trihydrate 3.68 g/L, Sodium Gluconate 5.02 g/L at pH 7.4)) thereby forming a dilute third human amniotic filtrate. This step is carried out if concentrations of the hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), and/or transthyretin are too high (too concentrated) and cannot achieve the concentration profile of the multi-part processed amniotic composition shown in FIG. 3, step (i).

Steps (a′)-(f) each occur below 21° C. to prevent and/or reduce any endogenous enzymatic degradative processes while preparing the filtrates disclosed therein. These steps occur in a sterile and/or aseptic environment, and as alluded to above, the third human amniotic filtrate (step (e′)) and/or the diluted third human amniotic filtrate (step (f)) is sterile, aseptic, and/or non-immunogenic.

After either step (e′) or (f), the micronized human amnion composition (formed during either step (f) or step (g) in FIG. 1) is mixed with the micronized human amnion composition with either the third human amniotic fluid retentate or the dilute third human amniotic filtrate thereby forming the multi-part processed human amniotic composition configured for treatment of Peyronie's disease by intracorporeal injection, wherein none of the steps include introduction of exogenous enzymes. In certain aspects and as shown in FIG. 3 step (i) as well as in FIGS. 5-9, 11 and 12, the multi-part processed human amniotic compositions includes HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, transthyretin ranging from 1.25×10⁴ to 2.25×10⁴ pg/mL and/or ranging from 1.3×10⁴ to 2.15×10⁴ pg/mL, or any combination thereof when the micronized human amnion composition is reconstituted, dispersed, and/or suspended in the aqueous human amnion filtrate. While in certain aspects, the multi-part processed human amniotic compositions includes HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5x106 pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, and transthyretin ranging from 1.25×10⁴ to 2.25×10⁴ pg/mL and/or ranging from 1.3×10⁴ to 2.15×10⁴ pg/mL when the micronized human amnion composition is reconstituted, dispersed, and/or suspended in the aqueous human amnion filtrate.

As further shown in step (j) of FIG. 3 and as discussed further below regarding the kits disclosed herein, the multi-part processed amniotic composition for treatment of Peyronie's disease is subsequently packaged in a sterile container (e.g., sterile vial, sterile ampule, or sterile pre-loaded syringe) for subsequent use for intracorporeal injection into the penis (tunica albuginea, corpus cavernosum, and/or plaque) to treat Peyronie's disease and/or minimize symptoms of Peyronie's disease. When packaged and/or stored within the sterile containers, the multi-part processed amniotic composition for treatment of Peyronie's disease is also sterile, aseptic, and/or contaminant free to minimize and/or prevent unwanted side effects and/or to minimize immunogenic responses within a subject during treatment with the disclosed compositions.

### Kits Containing Multi-Part Processed Amniotic Composition

Also disclosed herein are kits including the multi-part processed human amniotic composition configured for treatment of Peyronie's disease by intracorporeal injection in a subject in need thereof with an effective amount of the multi-part process human amniotic composition. The kit includes a sterile container having the multi-part processed human amniotic composition configured for treatment of Peyronie's disease by intracorporeal injection stored therein, wherein: the multi-part processed human amniotic composition configured for treatment of Peyronie's disease by intracorporeal injection comprises micronized human amnion composition suspended in an aqueous human amniotic fluid filtrate, but does not include any exogenous enzymes added thereto. For example, FIG. 4 is a photograph of the multi-part processed human amniotic composition packaged in an exemplary sterile container (100), stored at between −20° C. to −80° C. and ready for subsequent use. Other exemplary sterile containers may include ampules, pre-loaded syringes, and/or vials also stored at between −20° C. to −80° C. and ready for subsequent use. The kits, and more particularly the containers, disclosed herein may be configured for single use (e.g., preloaded syringe or ampule) or multi-use purposes (e.g., vial) and may include predetermined volumes therein, which range from 0.5 mL to 5 mL, from 1 mL to 4 mL, or from 1.5 to 3 mL in which any endpoint falling within these ranges may serve as endpoints for additional ranges. In certain aspects, the kits for single use may include the volume disclosed above (e.g., 0.5 mL, 0.75 mL, 1.0 mL, 1.25 mL, 1.5 mL, up to 5 mL) while the kits for multi-use may include volumes within the middle and upper endpoints of the above-mentioned ranges (e.g., 1.75 mL, 2.0 mL, 2.5 mL, 3.0 mL, 3.5 mL, 4.0 mL, 4.5 mL, and 5.0 mL). In certain aspects, the kits disclosed herein include the micronized human amnion composition and the aqueous human amniotic fluid filtrate are admixed at a ratio of 1 cm²: 1 ml to 2 cm²: 1 ml when forming the multi-part processed human amniotic composition. In certain aspects, the multi-part processed human amniotic compositions include a cryopreservant therein and at a concentration of 1 to 10 wt % of the overall concentration of the multi-part processed human amniotic compositions. For example, in certain aspects, the cryopreservant may be dimethyl sulfoxide (DMSO) in the multi-part processed human amniotic composition at an overall concentration of 1 to 10 wt % of the multi-part processed human amniotic composition. Alternatively, the cryopreservant may be a DMSO-free cryopreservant (e.g., a polyampholyte, P24, etc.) at an overall concentration of 1 to 10 wt % of the of the multi-part processed human amniotic composition.

### Methods of Using The Multi-Part Processed Amniotic Composition

Also disclosed herein are methods of treating Peyronie's disease in a subject in need thereof. These methods include (a) providing the multi-part processed human amniotic composition configured for treatment of Peyronie's disease; and (b) injecting the multi-part processed human amniotic composition configured for treatment of Peyronie's disease in an effective amount into the corpus cavernosum in the subject in need thereof thereby treating Peyronie's disease, reducing symptoms associated with Peyronie's disease, reducing plaque size associated with Peyronie's disease in the subject in need thereof, and/or improving and/or restoring penile function. In certain aspects, step(s) (a) and (b) as disclosed immediately above are repeated at predetermined time periods until the desired result is achieved (i.e., reducing symptoms associated with Peyronie's disease, reducing plaque size associated with Peyronie's disease in the subject in need thereof, and/or improving and/or restoring penile function).

In certain aspects, the method includes administering/injecting into the subject the multi-part processed human amniotic composition configured for treatment of Peyronie's disease by intracorporeal injection includes HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, transthyretin ranging from 1.25×10⁴ to 2.25×10⁴ pg/mL and/or ranging from 1.3×10⁴ to 2.15×10⁴ pg/mL, or any combination thereof and in an overall volume of 250 pL to 5 mL. In certain aspects, the method includes administering/injecting into the subject the multi-part processed human amniotic composition configured for treatment of Peyronie's disease by intracorporeal injection comprises HA at a concentration ranging from 1.2×10⁶ pg/mL to 1.1×10⁸ pg/mL and/or ranging from 1.3×10⁶ pg/mL to 5.5×10⁷ pg/mL, IGFBP-1 ranging from 2.5×10⁶ pg/mL to 9×10⁶ pg/mL and/or ranging from 2.75×10⁶ pg/mL to 5×10⁶ pg/mL, sGAGs ranging from 5×10⁷ pg/mL to 8.0×10⁷ pg/mL and/or ranging from 5.3×10⁷ pg/mL to 7.5×10⁷ pg/mL, exosomes having a particle diameter ranging from 50 to 200 nm and at a concentration ranging from 1.0×10⁹ pg/mL to 2.0×10⁹ pg/mL and/or ranging from 1.2×10⁹ pg/mL to 1.7×10⁹ pg/mL, IL-1ra ranging from 2×10⁴ pg/mL to 1.5×10⁵ pg/mL and/or ranging from 2.1×10⁴ pg/mL to 7×10⁴, pg/mL HGF ranging from 900 pg/mL to 3000 pg/mL and/or ranging from 1000 pg/mL to 1500 pg/mL, and transthyretin ranging from 1.25×10⁴ to 2.25×10⁴ pg/mL and/or ranging from 1.3×10⁴ to 2.15×10⁴ pg/mL and in an overall volume of 250 μL to 5 mL.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

1. A multi-part processed human amniotic composition configured for treatment of Peyronie's disease by intracorporeal injection in a subject in need thereof with an effective amount of the multi-part process human amniotic composition, the multi-part processed human amniotic composition comprising:
(a) a micronized human amnion composition; and
(b) an aqueous human amniotic fluid filtrate configured to reconstitute and suspend the micronized human amnion composition therein,
wherein:
the multi-part processed human amniotic composition are not processed with exogenous enzymes during production thereof and do not include exogenous enzymes added thereto.

2. (canceled)

3. The multi-part processed human amniotic composition configured for treatment of Peyronie's disease by intracorporeal injection according to claim 1, wherein the micronized human amnion composition has a particle diameter size ranging from greater than 1 μm to less than 300 μm.

4. (canceled)

5. (canceled)

6. The multi-part processed human amniotic composition configured for treatment of Peyronie's disease by intracorporeal injection according to wherein the micronized human amnion composition comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or any combination thereof.

7. (canceled)

8. The multi-part processed human amniotic composition configured for treatment of Peyronie's disease by intracorporeal injection according to claim 1, wherein the aqueous human amniotic fluid filtrate comprises hyaluronic acid (HA) and/or hyaluronan, fibronectin, insulin growth factor binding protein-1 (IGFBP-1), sulfated glycosaminoglycans (sGAGs), exosomes, interleukin-1 receptor antagonist (IL-1ra), hepatocyte growth factor (HGF), transthyretin, or a combination thereof.

9. The multi-part processed human amniotic composition configured for treatment of Peyronie's disease by intracorporeal injection according to claim 1, wherein the aqueous human amniotic fluid filtrate further comprises an isotonic solution.

10. (canceled)

11. The multi-part processed human amniotic composition configured for treatment of Peyronie's disease by intracorporeal injection according to claim 1, wherein the aqueous human amniotic fluid filtrate comprises particles that are less than 70 μm in diameter therein.

12. The multi-part processed human amniotic composition configured for treatment of Peyronie's disease by intracorporeal injection according to claim 1, wherein the composition comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ to 8.0×10⁷ pg/mL, exosomes having a particle diameter ranging from 50 to 200 nm and at a concentration and ranging from 1.0×10⁹ to 2.0×10⁹, IL-1ra ranging from 2×10⁴ to 1.5×10⁵ pg/mL, HGF ranging from 900 to 3000 pg/mL, transthyretin ranging from 1.25×10⁴ to 2.25×10⁴ pg/mL, or any combination thereof when the micronized human amnion composition is reconstituted and/or suspended in the aqueous human amnion filtrate.

13. (canceled)

14. The multi-part processed human amniotic composition configured for treatment of Peyronie's disease by intracorporeal injection according to claim 1, wherein the micronized human amnion composition and the aqueous human amniotic fluid filtrate are configured for admixing at a ratio of 1 cm²: 1 ml to 2 cm²: 1 ml.

15. (canceled)

16. (canceled)

17. (canceled)

18. (canceled)

19. (canceled)

20. A kit comprising a multi-part processed human amniotic composition of claim 1 configured for treatment of Peyronie's disease by intracorporeal injection in a subject in need thereof with an effective amount of the multi-part process human amniotic composition, the kit comprising:
a sterile container having the multi-part processed human amniotic composition configured for treatment of Peyronie's disease by intracorporeal injection stored therein,
wherein:
the multi-part processed human amniotic composition configured for treatment of Peyronie's disease by intracorporeal injection comprises micronized human amnion composition suspended in an aqueous human amniotic fluid filtrate, but does not include any exogenous enzymes added thereto.

21. The kit of claim 20, wherein collagenase is not included in the multi-part processed human amniotic composition configured for treatment of Peyronie's disease by intracorporeal injection.

22. (canceled)

23. The kit according to say one of claim 20, wherein the micronized human amnion composition has a particle diameter size ranging from greater than 1 μm to less than 300 μm.

24. (canceled)

25. (canceled)

26. (canceled)

27. (canceled)

28. (canceled)

29. (canceled)

30. The kit according to claim 1, wherein the aqueous human amniotic fluid filtrate further comprises an isotonic solution.

31. (canceled)

32. The kit according to claim 20, wherein the aqueous human amniotic fluid filtrate comprises particles that are less than 100 μm in diameter therein.

33. The kit according to claim 20, wherein the micronized human amnion composition and the aqueous human amniotic fluid filtrate are admixed at a ratio of 2:1 to 1:2.

34. The kit according to claim 20, wherein the multi-part processed human amniotic composition configured for treatment of Peyronie's disease by intracorporeal injection comprises HA at a concentration ranging from 1.2×10⁶ to 1.1×10⁸ pg/mL, IGFBP-1 ranging from 2.5×10⁶ to 9×10⁶ pg/mL, sGAGs ranging from 5×10⁷ pg/mL to 8.0×10⁷ pg/mL, exosomes having a particle diameter ranging from 50 to 200 nm and at a concentration ranging from 1.0×10⁹ to 2.0×10⁹, IL-Ira ranging from 2×10⁴ to 1.5×10⁵ pg/mL, HGF ranging from 900 to 3000 pg/mL, transthyretin ranging from 1.25×10⁴ to 2.25×10⁴ pg/mL, or any combination thereof.

35. (canceled)

36. (canceled)

37. (canceled)

38. (canceled)

39. (canceled)

40. (canceled)

41. A method of treating Peyronie's disease in a subject in need thereof, comprising:
(a) providing the multi-part processed human amniotic composition configured for treatment of Peyronie's disease of claim 1; and
(b) injecting the multi-part processed human amniotic composition configured for treatment of Peyronie's disease at an effective amount into a corpus cavernosum in the subject in need thereof thereby treating Peyronie's disease, reducing symptoms associated with Peyronie's disease, and/or reducing plaque size associated with Peyronie's disease in the subject in need thereof.

42. (canceled)

43. (canceled)

44. A method of making a multi-part processed human amniotic composition of claim 1 configured for treatment of Peyronie's disease by intracorporeal injection in a subject in need thereof, the method comprising:
(a) providing a fresh human amnion;
(b) separating the human amnion from other human placental components;
(c) rinsing the separated human amnion of step (b) with an alcohol;
(d) washing the separated human amnion of step (c) with an isotonic solution;
(e) drying separated human amnion of step (d) at ambient temperature for 2-6 hours in biosafety cabinet with circulating fan, or in a dehydrator for 15minutes to 1 hour at a temperature ranging from 30° C. to 40° C., thereby forming dried human amnion;
(f) grinding the dried human amnion thereby forming a micronized human amnion composition having particle diameters ranging from 1 to 500 microns;
(a′) providing a fresh human amniotic fluid;
(b′) filtering the fresh human amniotic fluid through a 200 micron filter thereby forming a first human amniotic fluid filtrate;
(c′) filtering the first human amniotic fluid filtrate through a 100 micron filter thereby forming a second human amniotic fluid filtrate;
(d′) filtering the second human amniotic fluid filtrate through a 70 micron filter thereby forming a third human amniotic fluid filtrate;
(f) optionally diluting the third human amniotic fluid filtrate in a predetermined amount of a balanced salt solution thereby forming a dilute third human amniotic filtrate;
(i) mixing the micronized human amnion composition with either the third human amniotic fluid filtrate or the dilute third human amniotic filtrate thereby forming the multi-part processed human amniotic composition configured for treatment of Peyronie's disease by intracorporeal injection, wherein none of the steps include introduction of exogenous enzymes.

45. (canceled)

46. The method of claim 44, further comprising, between steps (a)-(c), removing any blood clots present within the human amnion.

47. The method of claim 44, wherein step (d) is repeated between one to five times by discarding the used isotonic solution, and providing new isotonic solution and again washing the human amnion with the new isotonic solution.

48. (canceled)

49. The method of claim 44, wherein a grinding tool configured to grind and/or mince the dried human amnion is used during step (f) and grinds the dried human amnion at a range of 40 to 200 revolutions per minute (RPM) until the dried human amnion has been ground thereby forming the micronized human amnion composition having particle diameters ranging from 1 to 500 microns.

50. (canceled)

51. (canceled)

52. (canceled)

53. (canceled)
